(19) United States
(12) Patent Application Publication
(10) Pub. No.: US 2017/0367472 A1
(43) Pub. Date: Dec. 28, 2017

(54) ORAL TOOTHPASTE AND ELECTRICAL CLEANING DEVICE

(71) Applicant: BZL Medical Ltd., Kibbutz Habonim (IL)

(72) Inventor: BenZion Levi, Kibbutz Habonim (IL)

(73) Assignee: BZL Medical Ltd., Kibbutz Habonim (IL)

(21) Appl. No.: 15/629,769

(22) Filed: Jun. 22, 2017

Related U.S. Application Data
(60) Provisional application No. 62/353,172, filed on Jun. 22, 2016.

(51) Int. Cl. A46B 15/00; A46B 9/04; A61K 8/19; A61Q 11/00; A61C 17/22

(52) U.S. Cl. CPC A46B 15/0026; A61Q 11/00; A61C 17/225; A61K 8/19; A46B 9/04; A61C 17/224; A61C 17/221; A61K 2800/28

(57) ABSTRACT

An oral cleaning device includes a head portion that supports cleaning elements, a handle portion extending from the head portion, an EMF generator disposed in the handle portion. An inductor is located in the head portion and is connected to the EMF generator.

### FIELD OF THE INVENTION

The present invention relates generally to oral cleaning devices, and particularly to an oral cleaning device, such as a toothbrush, that generates a chemical agent in situ by applying electromagnetic field (EMF) energy to an inductor in the cleaning device in the presence of substances in the user's mouth.

### BACKGROUND OF THE INVENTION

Oral care products include a variety of chemical agents that perform a range of functions. For instance, toothpastes, mouthwashes and whiteners include chemical agents that help to clean teeth, kill bacteria, freshen breath and/or whiten teeth. These products typically cooperate with a device such as a toothbrush, an applicator device, or a tongue scraper, to perform their intended functions. In addition, chemical agents in these products may provide secondary benefits such as providing pleasing flavors or odors.

Oral care products that are directed to whitening teeth include an oxidizing agent as the primary active ingredient, such as hydrogen peroxide. These products are formulated into liquids, pastes or gels for application to the teeth. Upon storage, these products lose their whitening efficacy over time. In addition, these products have a brief period of efficacy when applied to the teeth in the oral cavity. For example, saliva contains high concentrations of the enzyme catalase, which on contact rapidly decomposes hydrogen peroxide into gaseous oxygen and water and so that there is only transitory contact of the peroxide whitening agent with the teeth. In addition, the low viscosities of aqueous peroxide solutions do not allow the peroxide whitening agent to remain in contact with the teeth for as long as is necessary to effect substantive whitening, because of the constant flushing effects of salivary secretions. As such, it is desirable to have high concentrations of oxidizing agents or effective oxidizing agents applied directly to teeth.

U.S. Pat. No. 8,156,602 to Jiminez et al., describes a device for generating a chemical agent in situ on an as-needed basis via the application of an electrical potential across a pair of conductors in communication with an electrolyte. The device is a toothbrush that generates chemical agents in a user's mouth by applying an electrical potential to an electrolyte such as saliva and/or a dentifrice located therein. The chemical agents include ozone, hydrogen peroxide, peroxide, chlorine and/or hypochlorite. The toothbrush includes a voltage source and a first set of electrodes for applying an electrical potential to the electrolyte. The toothbrush includes a second set of electrodes disposed about an anode of the first set of electrodes. The first and second sets of anodes cooperate to produce ions, peroxides, ozone and/or other chemical agents via the application of electrical potential to the electrolyte.

### SUMMARY OF THE INVENTION

The present invention seeks to provide an oral cleaning device, for generating a chemical agent in situ on an as-needed basis via the application of an electromagnetic field across an inductor. The device is a toothbrush that generates chemical agents in a user's mouth by applying an electromagnetic field to a dentifrice located therein. The dentifrice includes tiny iron filings.

There is thus provided in accordance with an embodiment of the present invention an oral cleaning device including a head portion that supports cleaning elements, a handle portion extending from the head portion, and an EMF generator disposed in the handle portion, which is connected to an inductor located in the head portion.

In accordance with an embodiment of the present invention a voltage source in the handle portion is connected to the EMF generator.

In accordance with an embodiment of the present invention the inductor is located along opposite sides of the head portion.

In accordance with an embodiment of the present invention the cleaning elements are located along opposite sides of the head portion.

In accordance with an embodiment of the present invention a dentifrice is provided that contains ferrous particles, such as tiny iron filings or a ferromagnetic polishing agent.

### BRIEF DESCRIPTION OF THE DRAWINGS

The present invention will be understood and appreciated more fully from the following detailed description, taken in conjunction with the drawings in which:

FIG. 1 is a perspective side view illustration of an oral cleaning device, e.g., a toothbrush, in accordance with a non-limiting embodiment of the invention.

FIG. 2 is a close-up, side view illustration of a bristle head portion including an inductor, in accordance with a non-limiting embodiment of the invention.

FIG. 3 is a close-up, side view illustration of a bristle head portion having an inductor and including a ferromagnetic core, in accordance with a non-limiting embodiment of the invention.

### DETAILED DESCRIPTION OF EMBODIMENTS

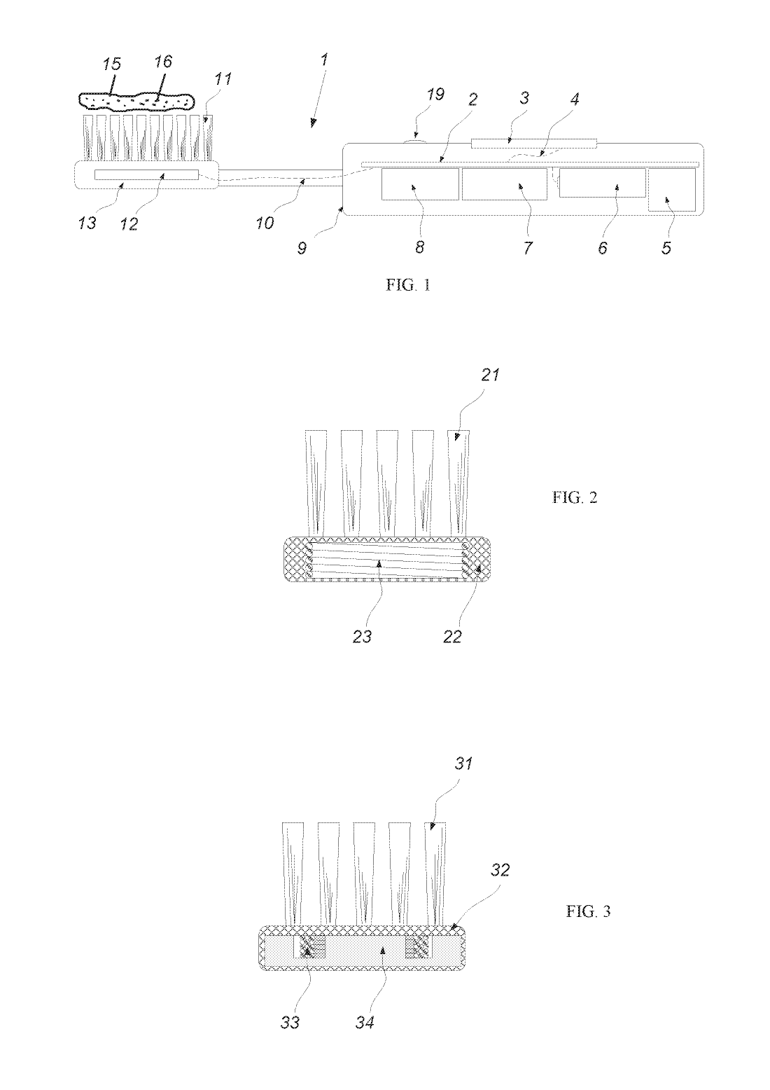

Reference is now made to FIG. 1, which illustrates an oral cleaning device 1, constructed and operative in accordance with a non-limiting embodiment of the present invention.

Device 1 includes a head portion 13 at one end supporting cleaning elements such as bristles 11, and a handle portion 9 at the opposite end. The handle portion 9 may house therein a voltage source 6, such as a battery (e.g., rechargeable). A battery charger 5 may also be provided in handle portion 9 for recharging battery 6. An EMF (electromagnetic field energy) generator 8 is provided in handle portion 9, powered by voltage source (battery) 6, for generating EMF energy in a frequency range, typically but not limited to, 10-500 Hz. All these components may be mounted on a printed circuit board 2 and operated with one or more control buttons 3 connected to PCB 2 with wires 4. The PCB 2 may also comprise control circuitry 7 for controlling the operation of the EMS generator 8 or the control circuitry may be built into EMS generator 8. One or more indicator lights 19 may be provided for indicating operating status and parameters to the user.

The EMF generator 8 is electrically connected to an inductor 12 (other configurations of inductors are shown in FIGS. 2 and 3) via EMF connecting wires 10. Inductor 12 may be located in the middle or along opposite sides of head portion 13. Bristles 11 may also be located along opposite sides of the head portion 13. The head portion 13 may be a replaceable case. More than one inductor 12 may be provided.

Inductor 12 is able to generate EMF within a user's mouth and generate chemical agents in situ at the application site of the agents. Device 1 may be used with a dentifrice 15 that includes ferrous particles 16, such as but not limited to, tiny iron filings, and/or a mixture of ferromagnetic tiny particles. Although shown as a toothbrush with a non-moving head, it is understood that device 1 could have a moving head and may have a variety of configurations.

The EMF generator 8 is electrically connected to inductor 12 for providing EMF energy to chemical agents within a user's mouth. The EMF energy enhances the efficacy of the chemical agents in the user's mouth.

Device 1 can generate the chemical agents in a variety of ways depending on factors such as the configuration of the toothbrush, the EMF energy (such as but not limited to, frequency 30 kHz and energy flux 0.8 mW/m²) provided by inductor or inductors 12 with or without ferromagnetic core or cores, the composition of the dentifrice, the composition of inductor 12 and other factors. For instance, in the presence of saliva alone, device 1 can generate hydrogen, oxygen, peroxide and ozone. In the presence of ionic compounds such as sodium chloride, potassium chloride, or calcium carbonate that may be contained in an aqueous dentifrice, chemical agents such as chlorine or calcium can be generated. In another example, in the presence of a dentifrice containing an activatable chemical agent that is stored in an inactive state, the chemical agent can be generated by being activated via the EMF energy.

The dentifrice may contain a gel material having dispersed therein a polishing agent. A polishing agent such as iron filings can be moved (linear and/or rotary motion) in the gel vehicle by EMF energy. The movement of the polish agent in the presence of substances in the user's mouth may provide cleaning and polishing functions. The iron filings can have a particle size of between 0.1 and 20 microns. Various other materials, such as surfactants, coloring agents, preservatives, antibacterial agents and others can be incorporated in the composition.

Many configurations of the EMF inductor and bristles (cleaning elements) can be made in accordance with the invention. Non-limiting examples are given in FIGS. 2-3.

FIG. 2 illustrates an embodiment of a head portion 22 with an air core inductor 23. Bristles 21 are located along opposite sides of the head portion 22.

FIG. 3 illustrates an embodiment of a head portion 32 with an inductor 33 mounted in a ferromagnetic core 34. Bristles 31 are located along opposite sides of the head portion 32.

What is claimed is:

1. An oral cleaning device comprising:
a head portion that supports cleaning elements;
a handle portion extending from said head portion;
an EMF (electromagnetic field energy) generator disposed in said handle portion; and
an inductor located in said head portion, connected to said EMF generator.

2. The oral cleaning device according to claim 1, further comprising a dentifrice composition comprising a ferrous material.

3. The oral cleaning device according to claim 1, comprising a voltage source in said handle portion connected to said EMF generator.

4. The oral cleaning device according to claim 1, wherein said inductor is located along opposite sides of said head portion.

5. The oral cleaning device according to claim 1, wherein said cleaning elements are located along opposite sides of said head portion.

6. The oral cleaning device according to claim 1, wherein said ferrous material comprises a ferromagnetic polishing agent.
